(19) United States
(12) Patent Application Publication
(10) Pub. No.: US 2007/0134277 A1
(43) Pub. Date: Jun. 14, 2007

(54) Pharmaceutical formulation for sulfur-containing drugs in liquid dosage forms

(75) Inventors: Wen G. Chen, Woburn, MA (US); Michael Shannon, Brookline, MA (US); Vimonrat Umprain, Sharon, MA (US); Whe-Yong Lo, Canton, MA (US); Karl P. Weinrich, Bridgewater, MA (US)

Correspondence Address: NIXON PEABODY, LLP, 401 9TH STREET, NW, SUITE 900, WASHINGTON, DC, 20004-2128, US

(73) Assignee: Children's Medical Center Corporation, Boston, MA (US)

(21) Appl. No.: 11/297,639

(22) Filed: Dec. 9, 2005

(51) Int. Cl. A61K 9/00; A61K 31/198

(52) U.S. Cl. 424/400; 514/562

(57) ABSTRACT

The pharmaceutical formulations of the invention for masking the odor from the sulfur-containing active agent comprise at least one sulfur-containing active agent, an effective amount of at least one flavoring agent. Any flavoring agent or combinations of flavoring agents may be used in the pharmaceutical formulation of the invention. The flavoring agent may be natural flavors, natural fruit flavors, artificial flavors, and mixtures thereof. The pharmaceutical formulation may contain an artificial sweetener, a natural sweetener or mixtures thereof. The pharmaceutical formulations are provided in liquid dosage form or a dry powder dosage form for reconstitution in water. Stabilizer added as one of expicients can extend the stability of the pharmaceutical formulation liquid dosage form for a period of at least 30 days when the formulation is stored below room temperature. The pharmaceutical formulations of the invention are palatable and particularly useful for the administration of sulfur-containing drugs to very small children that are in need of such medications. Methods of forming a liquid dosage form of pharmaceutical formulation by adding water to the dry powder form, methods to prepare an odor-masking pharmaceutical formulation and methods for treating lead poisoning or Wilson's disease using the odor-masking pharmaceutical formulation are also provided.

### FIELD OF THE INVENTION

The invention relates generally to pharmaceutical formulations of at least one sulfur-containing active agent, methods to prepare pharmaceutical formulations and methods to treat lead poisoning or Wilson's disease using the pharmaceutical formulations of the invention. More specifically, the invention relates to the pharmaceutical formulations comprising at least one sulfur-containing active agent, such as d-penicillamine, an effective amount of at least one flavoring agent to mask the odor from the sulfur-containing active agent. The pharmaceutical formulations may be in a dry powder form for reconstitution or in a liquid dosage form, such as an oral syrup.

### BACKGROUND

Penicillamine (3-mercapto-D-valine) is a chelating agent used in the treatment of Wilson's disease, also known as hepatolenticular degeneration. Penicillamine is approved by the United States Food and Drug Administration (“FDA”) as a treatment for Wilson's Disease, In the U.S. and Canada, d-penicillamine is commercially available under the trade names Cuprime and DEPEN®; Cuprime is available as 125 milligram (mg, 10⁻⁶ kilogram) and 250 milligram (mg) capsules and DEPEN® is available as scored 250 milligram (mg) tablets.

Wilson's Disease is a rare autosomal recessive inherited disease that causes excess copper accumulation primarily in the liver, brain, kidneys, and cornea; it affects about one in 30,000 people worldwide. While healthy people are able to excrete the copper they do not need, patients with Wilson's disease cannot. The liver of a person inflicted with Wilson's disease does not release copper into bile as it should; accordingly, copper builds up in the liver injuring the liver tissue. Eventually, the damage caused to the liver by the copper accumulation causes the liver to release the copper directly into the bloodstream, which carries the copper throughout the body. The copper buildup leads to damage in the kidneys, brain, and eyes. If left untreated, Wilson's disease can cause severe brain damage, liver failure, and death. Penicillamine removes excess copper from the affected tissues of patients with Wilson's disease. If the disorder is detected early and treated correctly, a person with Wilson's disease can enjoy completely normal health.

Penicillamine is also used for the treatment of severe, active rheumatoid arthritis unresponsive to conventional therapy and cystinuria to reduce cystine excretion and for prevention of the formation of kidney stones. See, e.g., U.S. Pat. No. 4,487,780 to Scheinberg and U.S. Pat. No. 4,680,309 to Maurer.

In addition to Wilson's disease, d-penicillamine is also used to treat lead poisoning in children. The use of d-penicillamine to treat lead poisoning is not approved by the FDA, but physicians, having found d-penicillamine to be an effective chelating agent to remove lead from afflicted tissues, have been using d-penicillamine to treat lead poisoned children for the past 30 years. Childhood lead poisoning, despite a dramatic fall in prevalence, continues to affect an estimated 310,000 children aged 1-5 years, or 1.6% of the U.S. population in that age range, have levels of lead in their bodies high enough to cause concern. (Blood Lead Levels-United States, 1999-2002, Centers for Disease Control and Prevention, MMWR, May 27, 2005/54(20); 513-516) Blood lead levels (BLL) in children found to be over 10 micrograms per deciliter, or 10 μg/dL, is considered as “elevated” and “unsafe.” (http://www.cdc.gov/nceh/lead/Publications/PrevLeadPoisoning.pdf).

Deteriorated lead-based paint in older homes and high levels of lead-contaminated house dust are the most common sources of lead poisoning in U.S. children. Lead paint is present in an estimated 24 million U.S. homes. More than 4 million of these are homes to one or more young children, according to the Centers for Disease Control and Prevention (CDC). (http://www.cdc.gov/nceh/lead/faq/about.htm) Sources of lead contamination may be from paint manufactured before 1977, pottery glaze, storage batteries, some solders, and some toys. Young children with blood lead levels above 10 μg/dL are at risk for a wide range of adverse neurodevelopmental effects, which may be outwardly manifested by cognitive losses, hyperactivity, impulsivity, aggression, and failure at school. Potential non-neurodevelopmental effects in lead-poisoned children consist of disturbances in heme synthesis and vitamin D activation as well as renal injury with an increased risk of adult hypertension.

Two oral chelators, d-penicillamine and succimer have been used to treat small children having lead poisoning through the daily administration of the agent by parents. d-penicillamine and succimer have similarities but also important differences. (Liebelt E. L. et al., Oral Chelators for childhood lead poinsing, Ped. Ann. 1994, 23:616-26) Both have an extremely unpleasant odor because of the high sulfur content of chelators. Succimer, approved in 1991 for the treatment of childhood lead poinsing, is prepared as a capsule which contains drug in a “sprinkle” form. The medication is easily placed on food or drink for administration to young children. d-penicillamine is only available as a tablet or capsulized powder, even though it has been used to treat lead poisoning by doctors more than 30 years. (Shannon M. W. et al., Efficacy of reduced-dose d-penicillamine in children with mild to moderate lead poisoning, Annals Phamacotherapy, 2000, 34:15-18; Piomelli S. et al., Management of Childhood lead poisoning, K. Pediatr. 1984, 105:523-32)

Unlike succimer, d-penicillamine absorption is significantly reduced by the presence of calcium so it is not given with a dairy product. With fewer options for concealing it in food or drink, d-penicillamine is considered to be more difficult to administer to children. The unpleasant odor of d-penicillamine renders the medication unpalatable and may lead to missed doses, frustrated parents, and extended treatment periods. Succimer can not completely substitute for d-penicillamine because succimer appears to be less effective in children with modest lead levels (blood lead<20-25 μg/dl) (Shannon M. W., et al., Efficacy of reduced-dose d-penicillamine in children with mild to moderate lead poisoning, Annals Phamacotherapy, 2000, 34:15-18; Piomelli S. et al., Management of Childhood lead poisoning, K. Pediatr. 1984, 105:523-32; Shannon M. W., Efficacy of d-penicillamine in children with small lead burdens, New Engl. J. Med. 1992).

The taste and odor masking of various medications has been addressed in the art of pharmaceutical preparation. Seventy to seventy-five percent of what is perceived as taste actually comes from the sense of smell. Taste buds can perceive only bitter, salty, sweet, and sour flavors. It's the odor molecules from food that give us most of our taste sensation. When the food is in the mouth, odor molecules from that food travel through the passage between the nose and mouth to olfactory receptor cells at the top of the nasal cavity, just beneath the brain and behind the bridge of the nose.

U.S. Pat. No. 5,494,681 to Cuca et al. describes a pharmaceutical delivery system comprising an active agent and a spatially oriented matrix comprised of a wax core having a melting point in the range of about 50° C. to about 200° C. and a regional hydrophobic material.

U.S. Pat. No. 5,728,403 to Mauger et al. describes a pharmaceutical coating for taste masking oral medications using a combination of triglycerides that melt at body temperature and a polymer that causes the coating to dissolve at pH 5.5.

U.S. Pat. No. 6,153,220 to Cummings et al. describes a taste-masking formulation for drugs having unpleasant organoleptic properties that uses cationic copolymers synthesized from dimethylaminoethyl methacrylate and neutral methacrylic acid esters in amounts significantly greater than the drug to form a taste-masked micromatrix powder that can be formed into dosage forms including sprinkles, suspensions, chewable tablets, fast melt tablets, and effervescent tablets.

U.S. Pat. No. 6,565,877 to Mukherji et al. describes a method to taste-mask bitter drugs by dissolving the active ingredient in methacrylic acid copolymer with phthalate polymer in a solvent and recovering the composition from the solution to form dry syrups, suspensions, conventional whole, chewable, or dispersible tablets. The Cuca et al, Mauger et al., Cummings et al., and Mukhedji et al., patents address only the masking of bitter-tasting drugs and do not address the masking of the odor of unpleasant drugs.

U.S. Pat. No. 6,159,504 to Kumabe describes calcium microparticles that may be used to cover a core substance, which is disclosed as including pharmaceuticals and which may be useful for, inter alia, masking the smell or bitter taste of the core substance.

U.S. Pat. Nos. 6,419,956 and 6,667,059, both to Sue et al. describes the masking of the odor as well as the taste, of Valerian Root tablets by covering the active agent with three coating compartments: the first coating comprising a hydroxymethylcellulose and an anti-tacking agent; the second coating comprising a sugar and at least one anti-tackiness agent; and the third coating comprising a methacrylate copolymer, a hydroxyalkyl cellulose, and an anti-tackiness agent.

While the Kumabe and Sue et al. patents discuss masking the smell of active agents, the patents are limited to doing so by surrounding the active agent in a tablet form with layers of materials.

However, because the administration of capsules or tablets to children is not always feasible, the coating methods described in the art have little benefit to mask the inherent odor of the sulfur-containing drugs. Accordingly, there remains a need in the art for the preparation of the pharmaceutical formulations to mask odor of the sulfur-containing agents, especially in liquid dosage foam, for the administration to pediatric patients.

In addition to the foregoing, because d-penicillamine has been shown to have poor stability in a liquid dosage form, prompt administration of d-penicillamine is imperative for proper treatment of patients requiring treatment. Therefore, there is a need to prepare pharmaceutical formulations of d-penicillamine in a liquid dosage form that has adequate stability for extended period of time as well as mask the pungent sulfur odor of d-penicillamine.

### SUMMARY OF THE INVENTION

The invention provides a pharmaceutical formulation comprising at least one sulfur-containing active agent, a method to prepare pharmaceutical formulation and a method to treat lead poisoning or Wilson disease using the pharmaceutical formulation. It has been found that the pharmaceutical formulation of the invention can mask the odor from the sulfur-containing agent and has excellent stability for extended period of time when in a liquid dosage form.

Accordingly, the pharmaceutical formulation comprising at least one sulfur-containing active agent, an effective amount of at least one flavoring agent to mask the odor from the sulfur-containing agent and combinations of excipients including at least one stabilizer to extend the stability of the pharmaceutical formulation when in a liquid dosage form represents one embodiment of the invention. The pharmaceutical formulation may be in a dry powder form for reconstitution or in a liquid dosage form.

The invention also provides a method of forming a liquid dosage form of a pharmaceutical formulation by adding water to solid dosage form and a method of odor-masking a pharmaceutical formulation comprising mixing at least one sulfur-containing active agent and at least one flavoring agent to mask the odor from the sulfur-containing agent.

In another aspect of the invention, there is provided a method to treat lead poisoning or Wilson's disease using an odor masking pharmaceutical formulation for the administration of d-penicillamine.

### DETAILED DESCRIPTION

Before describing the invention in detail it is to be understood that this invention is not limited to particular drug delivery systems, as such may vary. It is also to be understood that the terminology used herein is for the purpose of describing particular embodiments only, and is not intended to be limiting.

I. Definitions and Nomenclature

It must be noted that, as used in this specification and the appended claims, the singular forms “a,” “an” and “the” include plural referents unless the context clearly dictates otherwise. Thus, for example, reference to “a sulfur-containing active agent” includes a combination of two or more sulfur-containing active agent, reference to “a pharmaceutically acceptable carrier” includes combinations of two or more pharmaceutically acceptable carriers, and reference to “an excipient” includes combinations of two or more excipients.

In describing and claiming the invention, the following terminology will be used in accordance with the definitions set forth below.

A stereoisomer is a compound having the same molecular weight, chemical composition, and constitution as another, but with certain atoms arranged differently. That is, certain identical chemical moieties are at different orientations in space. This difference usually has the consequence of rotating the plane of polarized light in a differential manner. A pair of stereoisomers that are mirror images of each other are defined as enantiomers. Individual stereoisomers or enantiomers may have unique or beneficial properties that make that individual isomer particularly well suited for the invention. Consequently, individual stereoisomers or enantiomers and mixtures thereof of the active agents are included as part of the invention. Thus, each active agent may be present in the formulation as a racemate, i.e., equal amounts of each enantiomer, an enantiomerically pure form, or a mixture of nonequal amounts of each enantiomer.

The term “penicillamine” is used to refer to both isomers of penicillamine, that is, the d-isomer and the 1-isomer. For penicillamine, the d- and 1-isomers of the active agent are stereoisomers. Because 1-penicillamine has found to inhibit the action of pyridoxine (a B₆ vitamin) and hence is toxic in that respect, pharmaceutical formulations of penicillamine generally include only d-penicillamine. However, it is also understood that while d-penicillamine is presently used in commercially available forms of d-penicillamine, the possibility of using 1-penicillamine in the liquid pharmaceutical formulations described herein is also contemplated under the invention.

The terms “active agent,” “drug” and “pharmacologically active agent” are used interchangeably herein to refer to a chemical material or compound which, when administered to an organism (human or animal) induces a desired pharmacologic effect. Included are derivatives that include pharmacologically acceptable and pharmacologically active salts, esters and amides, as well as prodrugs and conjugates. Analogs of those compounds or classes of compounds specifically mentioned that also induce the desired pharmacologic effect, are also included.

The term “excipients” are substances other than the pharmacologically active drug or prodrug which are included in the manufacturing process or are contained in a finished pharmaceutical product dosage form. Excipients are classified by the functions they perform in a pharmaceutical dosage form. Principal excipient classifications (functions) are the following;binders disintegrants fillers (diluents), lubricants glidants (flow enhancers) compression aids colors sweeteners preservatives suspensing/dispersing agents film formers/coatings and flavors.

The term “pharmaceutically acceptable carrier” means a material, or materials, that are suitable for oral drug administration and are not biologically or otherwise undesirable, i.e., that may be administered to a patient along with an active agent without causing any undesirable biological effects or interacting in a deleterious manner with any of the other components of the pharmaceutical formulation in which it is contained. Typically, the material (e.g., carrier or excipient) has met the required standards of toxicological and manufacturing testing or it is included on the Inactive Ingredient Guide prepared by the U.S. Food and Drug administration. Similarly, a “pharmacologically acceptable” salt, ester, amide, or other derivative of an active agent as provided herein is a salt, ester, amide or other derivative that is not biologically or otherwise undesirable.

The term “dosage form” denotes any form of a pharmaceutical composition that contains an amount of active agent sufficient to achieve a therapeutic effect with a single administration. The frequency of administration that will provide the most effective results in an efficient manner without overdosing will vary with the characteristics of the particular active agent, including both its pharmacological characteristics and its physical characteristics, such as hydrophilicity. Within the context of the invention, the term “liquid dosage form” includes solutions, oral syrups, suspensions, and other liquid dosage forms known in the art. The term “dry powder for reconstitution” is understood to refer to reconstitution in a liquid such as water such that the resulting dosage form is a liquid dosage form, such as a solution, oral syrup, or suspension.

The term “patient” as in treatment of “a patient” refers to a mammalian individual afflicted with or prone to a condition, disease, or disorder as specified herein, and includes both humans and animals.

The terms “condition,” “disease,” and “disorder” are used interchangeably herein as referring to a physiological or pathophysiological state that can be prevented or treated by administration of a pharmaceutical formulation as described herein. For example, the pharmaceutical formulation of the invention may be used to prevent or treat patients suffering from or predisposed to Wilson's Disease, rheumatoid arthritis, cystinuria, kidney stone formation, or lead poisoning. As two examples, the pharmaceutical formulation of the invention may be used to treat persons who are predisposed to the formation of kidney stone or to treat persons that are clinically symptomatic and suffering from lead poisoning.

The term “treatment” as used herein refers to reduction in severity and/or frequency of symptoms, elimination of symptoms and/or underlying cause, prevention of the occurrence of symptoms and/or their underlying cause, and improvement or remediation of damage. Treatment of a patient suffering from a condition, disease, or disorder is accomplished by administering an “effective amount” or “therapeutically effective amount” of an active agent; the terms “effective amount” or “therapeutically effective amount” mean an amount of an active agent that is nontoxic, but sufficient to provide the desired therapeutic effect. The exact amount required will vary from subject to subject, depending on the age, weight, and general condition of the subject, the severity of the condition being treated, the judgment of the clinician, and the like. Thus, it is not always possible to specify an exact “effective amount”; however, an appropriate “effective” amount in any individual case may be determined by one of ordinary skill in the art using routine experimentation.

“Optional” or “optionally” means that the subsequently described event or circumstance may or may not occur, and that the description includes instances where said event or circumstance occurs and instances where it does not. For example, reference to an “optional” component in a formulation indicates that such a member may or may not be present, and the description includes formulations wherein a member is present and formulations wherein a member is not present.

Additional embodiments, advantages, and features of the invention will be set forth, in part, in the description that follows, and, in part, will become apparent to those skilled in the art upon examination of the following, or may be learned by practice of the invention.

II. Pharmaceutical Formulations

As noted above, the invention is directed to pharmaceutical formulations of sulfur-containing drugs, methods to prepare pharmaceutical formulations and methods to treat lead poisoning using the pharmaceutical formulations. The pharmaceutical formulations of the invention can mask the inherent odor of sulfur-containing agents of the sulfur-containing drugs and have excellent stability for extended period of time when in a liquid dosage form.

A. Pharmaceutically Acceptable Carriers

The pharmaceutical formulations of the invention may be in a dry powder form for reconstitution or in a liquid dosage form, such as an oral syrup. The techniques to prepare liquid dosage formulations and dry powder formulations for reconstitution in a liquid are well known in the art. See, for example, REMINGTON: THE SCIENCE AND PRACTICE OF PHARMACY, 20^{th} edition (Lippincott Williams & Wilkins, 2000).

For the dry powder formulations, the active agent, or combinations of active agents, is blended to form a substantially homogeneous powder mixture. Techniques involved with the preparation of such powders are well known in the art. The preparation of dry powder formulations often includes the steps of reducing the particle size of each active agent (again, alone or in combination), and blending. Reducing the particle size of each active agent is not required where a commercially available product having a suitable particle size is used. Techniques for reducing the particle size include, for example, using mills such as an air-jet mill or a ball mill. Similarly, the particle size of the remaining components (e.g., carrier, excipient, etc.) shall be controlled. The same techniques described above for reducing the particle size of active agents may be used to reduce the particle size of the remaining components. Again, such techniques are not required when the component is available commercially in the desired particle size range. Conventional blending techniques known to those skilled in the art may be used for combining one or more active agents with the remaining components. Such blending techniques include passing the combined powders through a sifter or blending, for example, the active agents and carrier in a powder blender such as a “double cone” blender or a “V-blender.” Regardless of the technique used, it is necessary that the resulting powder is a substantially homogeneous mixture.

After blending, the powder formulation may, if desired, be portioned and/or otherwise processed into unit dose quantities, e.g., portioned into unit dose quantities and individually placed within a unit dosage form or drug delivery system. Alternatively, the powder formulation may be loaded into a dosage form or drug delivery device and not “metered out” into unit doses until used.

The liquid dosage form of the pharmaceutical formulation of the invention is preferably a suspension or an oral syrup and may be prepared from a dry powder for reconstitution in water. The pharmaceutical formulations contain various excipients in order to mask the odor of the sulfur-containing agent of the sulfur-containing drugs. It is understood that the excipients that are to be used in the invention shall not have a deleterious effect on the intended patient or have a deleterious chemical or physical effect on any component in the composition. Thus, for example, excipients such as preservatives, surface active agents, buffering agents, suspending agents, and the like can be combined with the composition. The type and amount of any excipient will depend on the type of formulation used for administration, as will be appreciated by one of ordinary skill in the art. Specific examples of each of these excipients are well known by those skilled in the art of pharmaceutical formulation.

The referred formulations of the invention using d-penicillamine as the active agent are set forth in Tables 1, 2, 3, and 5 of Examples 1 to 4. The pharmaceutical formulation for sulfur-containing drugs in a liquid dosage form is also palatable. In order to ensure that the pharmaceutical formulations are palatable, sweeteners or sweeteners in combination of flavoring agents are used in appropriate amounts. The liquid dosage form of the pharmaceutical formulations of the invention are so effective at masking the offensive inherent odor of the sulfur-containing drugs contemplated under the invention that even toddlers, i.e., small children aged 1 to 3, accept the flavor-masked formulas, but not the unmasked formulas without flavorings (see, Example 2).

The pharmaceutical formulation should also include a suitable stabilizer. For example, because d-penicillamine is inherently unstable in aqueous solution and quickly forms the dimer d-penicillamine disulfide, which is considered to be a degradation product, the pharmaceutical formulation containing d-penicillamine also include a suitable stabilizer. Where the stability of the active agent in the pharmaceutical formulation is an issue, it is important that the excipients used in the pharmaceutical formulations of the invention do not have a deleterious effect on the stability of the pharmaceutical formulation. Table 5 of Example 4 presents a particularly preferred pharmaceutical formulation of the invention that includes d-d-penicillamine as the active agent and disodium dihydrate ethylenediaminetetraacetate (“EDTA”) as a stabilizer.

The presence of the sweeteners and mixtures with the flavoring agents in the pharmaceutical formulations ensure that they are palatable when administered, and the inclusion of a stabilizing agent, such as EDTA in the formulation ensures its stability for a period of at least 30 days when the pharmaceutical formulation is stored at proper temperature. (see, Example 4, Table 6). Preferably, the pharmaceutical formulations having penicillamine as the sulfur-containing active agent may be stored below room temperature. More preferably, the pharmaceutical formulations having penicillamine as the sulfur-containing active agent may be stored at refrigerator (−4° C.).

When the pharmaceutical formulations are in a liquid dosage form, it is preferred that the liquid is an aqueous solution, although aqueous suspensions may be used as well. The liquid dosage form of the pharmaceutical formulations may include one or more carriers in addition to the active agent. In addition to the carrier, the liquid dosage form of the pharmaceutical formulations may contain water and/or excipients including an antimicrobial preservative (e.g., methylparaben, propylparaben, butylparaben, benzalkonium chloride, benzethonium chloride, chlorobutanol, phenylethyl alcohol, sodium benzoate, thimerosal and combinations thereof), a buffering agent (e.g., citric acid, potassium metaphosphate, potassium phosphate, sodium acetate, sodium citrate, and combinations thereof), and/or a surfactant (e.g., Poloxamer, PEG 40 Stearate, polysorbate 80, sodium lauryl sulfate, sorbitan monopalmitate and combinations thereof). The pharmaceutical formulations may optionally contain a polymeric carrier and/or therapeutic extender, as described, for example, in U.S. Pat. No. 6,316,483, to Hanswalter et al.

B. Active Agents

Any of the active agents in the formulations may be administered in the form of a pharmacologically acceptable salt, ester, amide, prodrug, derivative, or as a combination thereof. Salts, esters and derivatives of the active agents may be prepared using standard procedures known to those skilled in the art of synthetic organic chemistry and described, for example, by J. March, ADVANCED ORGANIC CHEMISTRY: REACTIONS, MECHANISMS AND STRUCTURE, 4th Ed. (New York: Wiley-Interscience, 1992).

For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol and the acid is added at a temperature of about 0° C. to about 100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, and the like.

Preparation of esters involves functionalization of hydroxyl and/or carboxyl groups that may be present within the molecular structure of the drug. The esters are typically acyl-substituted derivatives of free alcohol groups, i.e., moieties which are derived from carboxylic acids of the formula RCOOH where R is alkyl, and preferably is lower, i.e., C₁₋₁₆ alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

The pharmaceutical formulations of the invention are not limited to the inclusion of one sulfur-containing drug, as combinations of sulfur-containing drugs may also be present. Sulfur-containing drugs contemplated as suitable for use in the pharmaceutical formulations and methods of the invention include by way of illustration, and not limitation, sulfur-containing metal chelating agents such as penicillamine, preferably d-penicillamine, and mercapthol. Sulfur-containing drugs that are not chelating agents contemplated under the invention include, without limitation, penicillins, cephalosporins, and piroxicam, sulfadiazine, sulfapyridine, and sulfathiazole. It is understood that within the context of the invention, the sulfur-containing drug may be present in the composition as a salt, ester, amide, prodrug, or other derivative, or may be functionalized in various ways as will be appreciated by those skilled in the art.

Preferably, the sulfur-containing active agent is a metal chelator. More preferably, the surfur-containing active agent is d-penicillamine. The d-penicillamine is preferably present in a range of about 0.1% w/w to about 20% w/w in the liquid dosage form, with a range of about 1% w/w to about 5% w/w more preferred. When the pharmaceutical formulation is prepared in a dry powder form for reconstitution, it is preferred that the d-penicillamine is present in a range of about 4% w/w to about 20% w/w. However, the weight percentage of the sulfur-containing drug in the claimed formulation may vary according to the sulfur-containing active agents.

C. Ingredients

To mask the inherent odor from the sulfur-containing agent and make the pharmaceutical formulation palatable, at least one flavoring agent is used in the pharmaceutical formulation of the invention. Any flavoring agent or combinations of flavoring agents may be used in the pharmaceutical formulation of the invention. The examples of the flavoring agents that may be used in the invention are natural flavors, natural fruit flavors, artifical flavors, and mixtures thereof. Preferably, the flavoring agent of the invention is OTTENS® flavorings. More preferably, the flavoring agent is a OTTENS® mixed berry flavor or OTTENS® grape flavor. The flavoring agents such as natural or artificial flavorings, may be present in the range of about 0.005% w/w to about 5% w/w, with a range of about 0.05% w/w to about 3% w/w preferred.

The pharmaceutical formulation may further comprise sweeteners or sweeteners in combination with other flavoring agents. The sweeteners may be an artificial sweetener, a natural sweetener or mixtures thereof. The sweeteners used may be selected from a wide range of materials including water-soluble sweeteners, water-soluble artificial sweeteners, water-soluble sweeteners derived from naturally occurring water-soluble sweeteners, and mixtures thereof. Without being limited to particular sweeteners, representative categories and examples are shown in Table I.

TABLE I

| Water-soluble Sweeteners | | Water-soluble Sweeteners |
|---|---|---|
| (monosaccharides, | | derived from naturally |
| disaccharides and | Water-soluble | occurring Water-soluble |
| polysaccharides) | Artificial Sweeteners | Sweeteners |
| xylose, ribulose, glucose | soluble saccharin salts, i.e., | chlorinated derivatives of |
| (dextrose), mannose, galactose, | sodium or calcium saccharin | ordinary sugar (sucrose), known, |
| fructose (levulose), sucrose (table | salts, cyclamate salts, the sodium, | for example under the produce |
| sugar), maltose, invert sugar (a | ammonium or calcium salt of | designation of sucralose. |
| mixture of fructose and glucose | 3,4-dihydro-6-methyl-1,2,3- | |
| derived from sucrose), partially | oxathiazine-4-one-2,2-dioxide, | |
| hydrolyzed starch, corn syrup | the potassium salt of 3,4-dihydro- | |
| solids, dihydrochalcones, | 6-methyl-1,2,3-oxathiazine-4- | |
| monellin, steviosides, | one2,2-dioxide (Acesulfame-K). | |
| glycyrrhizin, and sugar alcohols | | |
| such as sorbitol, mannitol, | | |
| xylitol, maltitol, hydrogenated | | |
| starch hydrolysates and mixtures | | |
| thereof. | | |

Preferably, the natural sweetener of the pharmaceutical formulation of the invention may be a sugar or sugar alcohol with examples selected from Table I. More preferred sugar-based sweeteners in the invention are dextrose, sucrose, and fructose, sorbitol, mannitol, xylitol and mixtures thereof.

Artificial/synthetic sweeteners, sugar alternatives, alternative sweeteners, non-nutritive sweeteners, non-caloric/low-cal/low-carb sweeteners, diabetic-safe sweeteners are all interchangeable and synonymous for the purposes of the invention. There are also a number of artificial sweeteners (polyols) available in the U.S., including erythritol, hydrogenated starch hydrosylates, isomalt, lacititol, maltitol, mannitol, sorbitol and xylitol. Polyols contribute between and 0.2 and three calories per gram as opposed to sucrose, which contributes four calories per gram. Polyols not only contribute sweetness but also bulk, and are used in a variety of products. Artificial sweeteners in the invention may include, but are not limited to, are sucralose, isomalt, aspartame, saccharin, lacititol, and other sweet replacers. Preferably, the artificial sweeteners in the pharmaceutical formulations of the invention are sodium saccharine, aspartame, lacititol, isomalt and sucralose.

The natural sweeteners may be present in a range of about 2% w/w to about 95% w/w, preferably with a range of about 50% w/w to about 95% w/w. Artificial sweeteners may be present in a range of about 0.01% w/w to about 2% w/w, with a range of about 0.05% w/w to about 1% w/w preferred.

In addition to flavorings, the pharmaceutical formulations of the invention may also contain the additional excipients. Preferred formulations of the invention with the additional excipients are set forth in Example 1. It is to be understood that the additional excipients set forth in Example 1 are representative of the preferred excipients that may be used with the invention and that other excipients may be used with the invention.

The pharmaceutical formulations of the invention may contain antimicrobial preservatives. Examples of such antimicrobial preservatives include benzalkonium chloride, cetylpyridinium chloride, cetylpyridinium bromide, chlorobutanol, chlorhexidine acetate, chlorhexidine HCl, chlorhexidine digluconate, chlorocresol, methylparaben, propylparaben, butylparaben, phenoxyethanol, phenylmercury salts, sodium benzoate, sorbic acid, and thiomersal. The antimicrobial preservatives used in the invention are generally in the range of about 0.001% w/w to about 2% w/w. In a preferred embodiment, the preservative system of the invention contains a combination of sodium methylparaben and propylparaben having a concentration of sodium methylparaben in a range of about 0.01% w/w to about 0.6% w/w, with a preferred range of about 0.005% w/w to about 0.2% w/w, and a concentration of propylparaben in a range of about 0.001% w/w to about 0.6% w/w, with a preferred range of about 0.0005% w/w to about 0.1% w/w. In another embodiment, the preferred preservative system of the invention contains sodium benzoate in a range of about 0.01% w/w to about 1.0% w/w with a preferred range of about 0.1% w/w to about 0.7% w/w.

The pharmaceutical formulations of the invention contain at least one stabilizer to help improve the stability of d-penicillamine when in a liquid dosage form. Examples of such stabilizers are ethylenediaminetetraacetate (EDTA) and its various salts. EDTA has been used as a stabilizing agent in the food industry. EDTA deactivates naturally-occurring enzymes by removing the metal ions from them and forming stable chelates with them. EDTA is also a versatile chelating agent by forming chelates with both transitional-metal ions. Calcium disodium EDTA exchanges its chelated calcium for lead and were first chelator in the treatment of lead poisoning. The resulting lead chealate is rapidly excreted in the urine. The calcium salt EDTA, when administered intravenously, is also used in the treatment of acute cadminum iron poisoning. A preferred stabilizer of the invention is disodium EDTA or disodium dihydrate EDTA.

The amount of EDTA as stabilizers for use in the invention are generally in the range of about 0.001% w/w to about 10% w/w, in a preferred range of about 0.05% w/w to about 5.0% w/w, with a range of about 0.1% w/w to about 2.0% w/w being more preferred. Because d-penicillamine is inherently unstable in solution, the liquid dosage formulations of d-penicillamine should be refrigerated to ensure that the formulation remains stable for the extended period of time, i.e., for a period of at least 30 days. Examples 3 and 4 show how the addition of an appropriate amount of EDTA may increase the stability of refrigerated liquid dosage formulations of d-penicillamine for a period in excess of 30 days.

The pharmaceutical formulation may optionally further comprises a pharmaceutical cosolvent, a dispersant, or a combination of the two. The examples of the cosolvents that may be used with the invention include, for example, propylene glycol, glycerin, water soluble polyethylene glycol (PEG) polymers and propylene glycol. The preferred cosolvent of the invention is PEG, which is commercially available in average molecular weights ranging from about 200 to greater than 20,000. The commercially available grades of PEG are marketed based on the average molecular weight, i.e., the grade nomenclature is identified with the molecular weight. For example, PEG 400 represents material with an average molecular weight of 400 and the material with an average molecular weight of 600 is known as PEG 600. PEG 200, 300, 400, and 600 are clear viscous liquids at room temperature, while PEG 900, 1000, 1450, 3350, 4500 and 8000 are white, waxy solids. Preferred PEGs for the pharmaceutical formulations of the invention are the short to medium chain PEG polymers such as PEG 400 to PEG 3350, with the most preferred PEG being PEG 400. The amount of cosolvent that may be used in the invention is in a range of about 1% w/w to about 30% w/w, with a preferred range of about 5% w/w to about 15.5% w/w. In pharmaceutical formulations where the active agent is d-penicillamine, the cosolvents should be PEG and similar cosolvents, such as propylene glycol, as glycerin is not compatible with d-penicillamine. Where the active agent is a metal chelator such as d-penicillamine, it is preferred that the cosolvent is propylene glycol or polyethylene glycol preferably present in a range of about 1% w/w to about 30% w/w in the liquid dosage form, with a range of about 5% w/w to about 15.5% w/w more preferred.

Dispersants that may be used with the invention include, for example, Carbopol, methylcellulose, hydroxypropylmethylcellulose (HPMC), hydroxyethylcellulose, hydroxypropylcellulose, sodium carboxymethylcellulose, polyvinyl alcohol, polyvinylpyrrolidone, polyacrylates, polyacrylamide, dextran, gellan gum, poloxamer, calcium polycarbophil, cellulose acetate phthalate, sodium hyaluronate, hyaluronic acid, alginate, chitosan, and so forth. The amount of dispersant that may be used in the invention is in a range of about 0.01% w/w to about 10% w/w, with a preferred range of about 0.1% w/w to about 1% w/w. In pharmaceutical formulations where the active agent is d-penicillamine, the preferred dispersant is HPMC.

The pharmaceutical formulations of the invention can also optionally include pharmaceutically acceptable buffering agents sufficient to adjust and maintain the pH of the liquid dosage form of the invention in the range of about 2.0 to about 7.0, preferably about 4.0 to about 5.0. Suitable buffering agents include citrate, phosphate, tromethamine, glycine, borate, or acetate salts, which can also be derived from substances of the type such as citric acid, primary or secondary sodium phosphate, glycine, boric acid, sodium tetraborate, acetic acid, and sodium acetate. Where appropriate, the pH of the liquid dosage form of the pharmaceutical formulation may be adjusted with the addition of a suitable acid such as citric acid, phosphoric acid, succinic acid, or tartaric acid in a quantity suitable to achieve a pH in the range of 2.0 to 7.0. Hydrochloric acid or sodium hydroxide can also be used for pH adjustment.

III. Utility and Administration

The invention has utility in that it allows otherwise unpalatable drugs to be administered with little to no discomfort for a time period sufficient to complete a treatment regime. The liquid dosage form of the pharmaceutical formulation such as a flavored oral syrup can be easily administered to children, especially to young children. Because one of the uses of the invention is for administration of d-penicillamine to treat lead poisoning in very small children, the pharmaceutical formulation was designed to be as appealing as possible to small children and in a mode of administration that is easily administrable to small children.

Although a primary use of the invention is the administration of otherwise unpalatable drugs to small children, it is understood that the invention will certainly also benefit adults, who should not be forced to suffer unpalatable medications when they do not have to. It is also understood that the invention is useful not only for the administration of d-penicillamine, it is also useful for the administration of any unpalatable drug, in particular sulfur-containing drugs, such as metal chelators.

Because the pharmaceutical formulation includes a stabilizer to protect the stability of the sulfur-containing drugs when in a liquid dosage form, the pharmaceutical formulation according to the method described herein have a shelf life of at least 30 days, and even beyond, when in properly stored below room temperature conditioned temperatures. The extended shelf life of the pharmaceutical formulation is particularly useful where the pharmaceutical formulation includes d-penicillamine as an active agent and is used for lead chelation therapy. It is understood that continued or multiple treatment may be necessary during the course-of treatment although treatment regime typically lasts for approximately three weeks.

All patents and publications mentioned herein are hereby incorporated by reference in their entireties.

The following examples are put forth so as to provide those of ordinary skill in the art with a complete disclosure and description of how to make and use the compositions of the invention. It is to be understood that while the invention has been described in conjunction with the preferred specific embodiments described above, the foregoing descriptions of the invention as well as the examples that follow are intended to illustrate and not limit the scope of the invention. Other aspects, advantages, and modifications within the scope of the invention will be apparent to those skilled in the art to which the invention pertains.

### EXPERIMENTAL

The practice of the invention as set forth in the examples, will use, unless otherwise indicated, conventional techniques of pharmaceutical formulation and medicinal chemistry that are within the skill level of those in the art. Preparation of various types of pharmaceutical formulations are described, for example, in REMINGTON: THE SCIENCE AND PRACTICE OF PHARMACY, 20^{th} edition (Lippincott Williams & Wilkins, 2000) and Ansel et al., PHARMACEUTICAL DOSAGE FORMS AND DRUG DELIVERY SYSTEMS, 6^{th} Ed. (Media, Pa.: Williams & Wilkins, 1995).

In the following examples, efforts have been made to ensure accuracy with respect to numbers (e.g., amounts, temperature, etc.) but some experimental error and deviations should, of course, be allowed for. Unless indicated otherwise, parts are parts by weight (w/w), temperature is degrees centigrade (° C.) and pressure is at or near atmospheric. All components were obtained commercially unless otherwise indicated. Unless otherwise indicated, all formulations described herein were performed with commercially available products.

### EXAMPLE 1

### Formulation for D-Penicillamine Oral Syrup or Dry Power for Reconstitution

Ranges and preferred ranges for preparation of the d-penicillamine oral syrup or dry powder for reconstitution of the invention are set forth in Table 1.

TABLE 1

| | | PREFERRED |
|---|---|---|
| INGREDIENT | RANGE | RANGE |
| d-penicillamine | 0.1% to 20% | 1% to 5% |
| artificial sweetener | 0.01% to 2% | 0.05% to 1% |
| natural sweetener | 2% to 95% | 50% to 95% |
| natural or artificial flavor | 0.005% to 5% | 0.05% to 3% |
| sodium methylparaben | 0.01% to 0.6% | 0.005% to 0.2% |
| propylparaben | 0.001% to 0.6% | 0.005 to 0.1% |
| (preservative system) | | |
| sodium benzoate | 0.01% to 1.0% | 0.1% to 0.7% |
| (alternative preservative) | | |
| EDTA | 0.001% to 10% | 0.05% to 5% |
| (used as a stabilizer) | | |
| PEG or propylene glycol | 1% to 30% | 5% to 15.5% |
| (used as a pharmaceutical | | |
| cosolvent) | | |
| HPMC | 0.01% to 10% | 0.1% to 1% |
| (used as a dispersant) | | |
| purified water USP | q.s. | q.s. |

### EXAMPLE 2

### D-Penicillamine Formulation Odor Test

The olfactory stimulation of the pharmaceutical formulation of the invention with d-penicillamine as an active agent was tested on 12 children aged 1 to 3 years. The d-penicillamine formulations administered to the toddlers are set forth in Table 2 and include one placebo with no flavoring (Formula A), one formulation with OTTENS® Mixed Berry flavoring (Formula B), and one formulation with OTTENS® Concord Grape flavoring (Formula C). Because the formulation was being tested only for odor and not for palatability, to increase the stability of the formulation for the duration of the Odor Test, sucrose was not included in the pharmaceutical formulation.

TABLE 2

| INGREDIENT | AMOUNT |
|---|---|
| d-penicillamine | 2.5% |
| sodium saccharine | 0.2% |
| disodium EDTA | 0.05% |
| Flavorings | |
| Formula A: Placebo | 0.0% |
| Formula B: OTTENS ® Mixed Berry | 1% |
| Formula C: OTTENS ® Concord Grape | 1% |
| sodium methylparaben | 0.155% |
| propylparaben | 0.0168% |
| phosphoric acid | to pH 4.0 |
| purified water USP | q.s. |

Children aged 1 to 3 years, the most common age for lead poisoning, were the subjects for the study; the children were accompanied by their parents. The children in the study were not exposed to the formulations other than to smell the formulations and determine their like/dislike for the odor of the formulation. Since children at such a young age are generally unable to articulate preferences between olfactory stimuli, the parents were instructed to act as interpreters for the child's response. The formulation order was pre-established as A/B, A/C, B/A, B/C, C/A, or C/B. The parents were asked to have the child smell two of the formulations followed by their qualitative interpretation of the child's response and their assessment of which aroma the child preferred. The formulations were ranked in order of preference. The results of the study found that Formulation B, which contained the Grape flavoring, was the most preferred of the three formulations.

### EXAMPLE 3

### Stability Evaluation of D-Penicillamine Powder for Reconstitution

Two formulations of d-penicillamine were prepared to determine whether a preservative system might have an effect on both powder stability and reconstituted solution stability of the pharmaceutical formulation. The formulations are set forth in Table 3.

TABLE 3

| INGREDIENT | FORMULA 1 | FORMULA 2 |
|---|---|---|
| d-penicillamine | 10.78% | 10.78% |
| saccharin sodium | 0.86% | 0.86% |
| sorbitol | 86.22% | 86.44% |
| OTTENS ® Grape flavor | 0.30% | 0.30% |
| sodium methylparaben | 0.67% | 0.00% |
| propylparaben | 0.07% | 0.00% |
| sodium benzoate | 0.00% | 0.52% |
| disodium dihydrate EDTA | 0.22% | 0.22% |
| citric acid | 0.89% | 0.89% |
| Total | 100.00% | 100.00% |

To reconstitute the powder, 23.2 g of powder was dissolved q.s. in deionized water to obtain a solution containing 25 mg/mL of d-penicillamine. The three solutions obtained from the formulations in Table 3 were placed in a refrigerator and samples were drawn and evaluated after 7 days, 14 days, 21 days, and 28 days for the presence of d-penicillamine disulfide. As there were no previous data using sodium benzoate, two containers were tested for Formula 2. The results of the study are shown in Table 4.

TABLE 4

| | % D-PENICILLAMINE DISULFIDE | | |
|---|---|---|---|
| | | FORMULA 2 | FORMULA 2 |
| TIME | FORMULA 1 | (CONT 1) | (CONT 2) |
| 0-Time | 0.23 | 0.13 | 0.14 |
| 7-days | 1.68 | 1.12 | 0.49 |
| 14-days | 2.06 | 2.30 | 1.35 |
| 21-days | 3.66 | 4.05 | 1.03 |
| 28-days | 4.73 | 6.54 | 1.92 |

The discrepancy between Container 1 and Container 2 of Formula 2 was obvious. In considering the cause for the increase in d-penicillamine disulfide from Container 1 versus Container 2 of Formula 2, it was theorized that perhaps it was possible that Container 1 had more EDTA than did Container 2; this theory was tested as set forth in Example 4.

### EXAMPLE 4

### Effect of EDTA Levels on Formulation Stability

To determine whether EDTA levels may have an impact on the stability of d-penicillamine in solution, three formulations containing d-penicillamine were prepared with increasing amounts of EDTA; the three formulations are set forth in Table 5.

TABLE 5

| INGREDIENT | FORMULA A | | FORMULA B | | FORMULA C | |
|---|---|---|---|---|---|---|
| d-penicillamine | 2.5109 | g | 2.4977 | g | 2.4978 | |
| saccharin sodium | 0.2007 | g | 0.2002 | g | 0.2001 | g |
| sorbitol | 20.0412 | g | 20.0562 | | 20.0522 | |
| OTTENS ® Grape | 0.0705 | g | 0.0710 | g | 0.0703 | g |
| flavor | | | | | | |
| sodium benzoate | 0.1239 | g | 0.1214 | g | 0.1222 | g |
| disodium dihydrate | 0.0503 | g | 0.1005 | g | 0.1539 | g |
| EDTA | | | | | | |
| citric acid | 0.2070 | g | 0.2065 | g | 0.2066 | g |
| deionized water | q.s. to 100 | mL | q.s. to 100 | mL | q.s. to 100 | mL |

The three solutions obtained from the formulations in Table 5 were placed into a refrigerator and samples were drawn and evaluated at 0 days, 7 days, 18 days, and 33 days for the presence of d-penicillamine disulfide. The results of the study are shown in Table 6.

TABLE 6

| | % D-PENICILLAMINE DISULFIDE | | |
|---|---|---|---|
| TIME | FORMULA A | FORMULA B | FORMULA C |
| 0-time | 0.18% | 0.18% | 0.17% |
| 7-days | 1.4% | 0.8% | 0.3% |
| 18-days | 3.0% | 1.7% | 1.2% |
| 33-days | 5.8% | 2.7% | 1.8% |

As shown in Table 6, Formula C, which had the highest amount of EDTA, showed the lowest percentage of d-penicillamine disulfide at all time points greater than 0-time. The results of Table 6 clearly demonstrate that increased levels of EDTA help to protect against the formation of d-penicillamine disulfide degradation product in d-penicillamine pharmaceutical formulations.

The claimed invention is:

1. A pharmaceutical formulation comprising:
at least one sulfur-containing active agent; and
an effective amount of at least one flavoring agent to mask odor from the sulfur-containing active agent.

2. The pharmaceutical formulation of claim 1, wherein the flavoring agent is selected from the group consisting of a natural flavor, an artificial flavor, and mixtures thereof.

3. The pharmaceutical formulation of claim 2, further comprising a natural sweetener or an artificial sweetener.

4. The pharmaceutical formulation of claim 3, wherein the natural sweetener is selected from the group consisting of sucrose, dextrose, fructose, sorbitol, xylitol, mannitol and mixtures thereof and the artificial sweetener is selected from the group consisting of sodium saccharine, aspartame, lacititol, isomalt, sucralose and mixtures thereof.

5. The pharmaceutical formulation of the claim 2, wherein the flavoring agent is a mixed berry flavor or a grape flavor.

6. The pharmaceutical formulation of claim 1, wherein the formulation is in a palatable liquid dosage form.

7. The pharmaceutical formulation of claim 6, wherein the liquid dosage form is a solution, suspension, or an oral syrup.

8. The pharmaceutical formulation of claim 6, further comprising a pharmaceutically acceptable excipient selected from the group consisting of a stabilizer, a preservative, a buffering agent, or a mixture thereof.

9. The pharmaceutical formulation of claim 8, wherein the excipient when present in the liquid dosage form is present in the amount of:
0.001% w/w to 10% w/w of a stabilizer;
0.001% w/w to 1.0% w/w of a preservative;
an effective amount of a buffer to maintain a pH range between pH 2 and pH 7 upon reconstitution into a liquid dosage form;
or as a mixture thereof.

10. The pharmaceutical formulation of claim 8, wherein the excipient when present in the liquid dosage form is present in the amount of:
0.05% w/w to 5% w/w of a stabilizer;
0.005% w/w to 0.7% w/w of a preservative;
an effective amount of a buffer to maintain a pH range between pH 4 and pH 5 upon reconstitution into a liquid dosage form;
or as a mixture thereof.

11. The pharmaceutical formulation of claim 8, wherein:
the stabilizer is disodium EDTA or disodium dihydrate EDTA;
the preservative is sodium methylparaben, propylparaben, sodium benzoate or mixtures thereof; and
the buffer is selected from the group consisting of citric acid, phosphoric acid, succinic acid, and tartaric acid.

12. The pharmaceutical formulation of claim 8, further comprising about 1% w/w to about 30% w/w of a pharmaceutical cosolvent and optionally, 0.01% w/w to 10% w/w of a dispersant in the liquid dosage form.

13. The pharmaceutical formulation of claim 12, wherein the pharmaceutical cosolvent is propylene glycol or polyethylene glycol and the optional dispersant is hydroxypropylmethylcellulose (HPMC).

14. The pharmaceutical formulation of claim 12, wherein the pharmaceutical cosolvent is present in a range of about 5% w/w to about 15.5% w/w and optionally, 0.1% w/w to 1% w/w of a dispersant in the liquid dosage form.

15. The pharmaceutical formulation of claim 12, wherein the excipient when present in the liquid dosage form is present in the amount of:
0.001% w/w to 10% w/w of a stabilizer;
0.001% w/w to 1.0% w/w of a preservative;
an effective amount of a buffer to maintain a pH range between pH 2 and pH 7 upon reconstitution into a liquid dosage form;
or as a mixture thereof.

16. The pharmaceutical formulation of the claim 6, wherein the pharmaceutical formulation in the liquid dosage form stays stable for a period of at least 30 days when the formulation is stored below room temperature.

17. The pharmaceutical formulation of claim 6, wherein the sulfur-containing active agent is a metal chelator.

18. The pharmaceutical formulation of claim 17, wherein the metal chelator is d-penicillamine and the d-penicillamine is present in a range of about 0.1% w/w to about 20% w/w in the liquid dosage form.

19. The pharmaceutical formulation of claim 18, wherein the d-penicillamine is present in a range of about 1% w/w to about 5% w/w in the liquid dosage form.

20. The pharmaceutical formulation of claim 1, wherein the formulation is in a dry powder form for reconstitution into a liquid dosage form.

21. The pharmaceutical formulation of the claim 20, wherein the flavoring agent is selected from the group consisting of a natural flavor, an artificial flavor, and mixtures thereof.

22. The pharmaceutical formulation of claim 21, further comprising a natural sweetener or an artificial sweetener.

23. The pharmaceutical formulation of claim 20, further comprising a pharmaceutically acceptable excipient selected from the group consisting of a stabilizer, a preservative, a buffering agent, or a mixture thereof.

24. The pharmaceutical formulation of claim 23, wherein the excipient when present in the liquid dosage form is present in the amount of:
0.001% w/w to 10% w/w of a stabilizer;
0.001% w/w to 1.0% w/w of a preservative;
an effective amount of a buffer to maintain a pH range between pH 2 and pH 7 upon reconstitution into a liquid dosage form;
or as a mixture thereof.

25. A method of forming a liquid dosage form of a pharmaceutical formulation by adding water to the dry powder form of claim 20.

26. The method of claim 25, wherein the liquid dosage form of a pharmaceutical formulation further comprising about 1% w/w to about 30% w/w of a pharmaceutical cosolvent.

27. The method of claim 25, wherein the liquid dosage form of a pharmaceutical formulation having a d-penicillamine as a sulfur-containing active agent and the d-penicillamine is present in a range of about 0.1% w/w to about 20% w/w in the liquid dosage form.

28. A method of odor-masking a pharmaceutical formulation comprising:
mixing at least one sulfur-containing active agent; and
an effective amount of at least one flavoring agent to mask odor from the sulfur-containing active agent.

29. The method of claim 28, wherein the flavoring agent is selected from the group consisting of a natural flavor, an artificial flavor, and mixtures thereof.

30. The method of claim 29, further comprising a natural sweetener or an artificial sweetener.

31. The method of claim 28, further comprising a step of mixing a pharmaceutically acceptable excipient with at least one sulfur-containing active agent and an effective amount of at least one flavoring agent to mask odor from the sulfur-containing active agent, wherein the pharmaceutically acceptable excipient is selected from the group consisting of a stabilizer, a preservative, a buffering agent, or a mixture thereof.

32. A method for treating lead poisoning or Wilson's disease comprising the step of administering to a patient in need thereof an odor masking a pharmaceutical formulation comprising:
at least one sulfur-containing active agent; and
an effective amount of at least one flavoring agent to mask odor from the sulfur-containing active agent.

33. The method of claim 32, wherein the flavoring agent is selected from the group consisting of a natural flavor, an artificial flavor, and mixtures thereof.

34. The method of claim 33, wherein the pharmaceutical formulation further comprising a natural sweetener or an artificial sweetener.

35. The method of claim 32, wherein the formulation is in a palatable liquid dosage form.

36. The method of claim 35, wherein the pharmaceutical formulation further comprising a pharmaceutically acceptable excipient selected from the group consisting of a stabilizer, a preservative, a dispersant, a buffering agent, or a mixture thereof.

37. The method of claim 36, wherein the excipient-when present in the liquid dosage form is present in the amount of:
0.001% w/w to 10% w/w of a stabilizer;
0.001% w/w to 1.0% w/w of a preservative;
an effective amount of a buffer to maintain a pH range between pH 2 and pH 7 upon reconstitution into a liquid dosage form;
or as a mixture thereof.

38. The method of claim 36, wherein the pharmaceutical formulation further comprising about 1%w/w to about 30% w/w of a pharmaceutical cosolvent and optionally, 0.01% w/w to 10% w/w of a dispersant in the liquid dosage form.

39. The method of claim 32, wherein the sulfur containing active agent is d-penicillamine and the d-penicillamine is present in a range of about 0.1% w/w to about 20% w/w in the liquid dosage form.
